(19) United States
(12) Patent Application Publication
(10) Pub. No.: US 2012/0237596 A1
(43) Pub. Date: Sep. 20, 2012

(54) NUTRIENT DELIVERY DRUG COMPOSITION

(76) Inventor: Paul Daniel Yered, Thousand, CA (US)

(21) Appl. No.: 13/484,046

(22) Filed: May 30, 2012

Related U.S. Application Data
(63) Continuation of application No. 13/038,390, filed on Mar. 2, 2011.

(51) Int. Cl. A61K 31/4545; A61P 29/00; A61K 9/48

(52) U.S. Cl. 424/451; 514/290

(57) ABSTRACT

The present invention is a chemical composition that includes loratidine, a selected one or more of an expeller pressed and a cold pressed oil, a selected one or more of a non-genetically modified, a non-genetically engineered and a non-chemically treated starch and a selected one or more of a non-genetically modified, a non-genetically engineered and a non-chemically treated cellulose. The chemical composition can also include a vitamin E oil preservative and infused chili olive oil and is typically encased in a capsule.

This application claims priority to U.S. Provisional Application 61/311,324 filed on Mar. 6, 2010, the entire disclosure of which is incorporated by reference. The present application is a continuation of U.S. Non-Provisional patent application Ser. No. 13/038,390 filed on Mar. 2, 2011, the entire disclosure of which is incorporated by reference.

### TECHNICAL FIELD & BACKGROUND

Most prescription drugs and many over the counter products contain excipients that may also be referred to as inactive ingredients, such as polysaccharides and other artificial additives. Many of these inactive ingredients are commonly added to foods by the food industry. For example, many foods contain artificial flavors, dyes, and other additives such as Xanthan Gum and various sugars, which are also found in medications. Excipients and other inactive ingredients are sometimes needed to deliver a drug with an active ingredient to the site of drug interaction when taken orally, whether to facilitate dissolution, absorption, distribution and excretion, while intravenous drugs do not require these inactive ingredients and therefore show relatively better and quicker results. The ratio of a drug and it's excipients in addition to any food additives ingested may be important with regard to the efficacy of a drug, considering many excipients are the same as or directly or indirectly related to many food additives.

These drug additives may result in unwanted impurities and consequences. Metformin, for example, is meant to lower a diabetic's blood sugar, but at the same time, contains polysaccharides which can have the opposite effect. Warfarin is meant to thin the blood but contains modified cornstarch, which also has the opposite effect. Furthermore the inactive ingredients may form unwanted complexes with or without artificial food additives after continued administration. The processing of excipients may contribute impurities and possibly toxins to the medications. For example, many starches and polysaccharides are washed with chlorine (hypochlorite), dextrins, phosphates and other synthetic acids and organisms.

The medical field continues to recognize the need for improving results from drug therapies, in addition to improving the efficacy of each drug. The obvious advantage is to decrease the amount and time at which the patient must be prescribed the drug to minimize any adverse effects that may occur. A novel way for making the medications potentially more effective and healthier would be to include excipients that are pure, natural and potentially nutritious. More importantly the patient is exposed to less chemically treated and modified polysaccharides and more nutrients that have not been modified.

Previously ingested food additives resulting in incubated and/or fermented stored complexes may further result in free radical toxin byproducts when exposed to re-administered excipients and/or artificial food additives. These previously ingested food additives may also contribute to artificially induced tissue layers and unwanted inactive ingredient group (IIG) complexes that may remain stored and/or latent for additional incubation and/or fermentation. The continued modified and attenuated food additive and inactive ingredient complexes may also continuously be re-activated and re-stored. Many foods contain chemicals and/or toxins such as food additives. It is well known that many of these food chemicals have been shown to be carcinogenic (i.e. Saccharin and yellow dye 6). Many of these are referred to as food additives, artificial additives, artificial flavors, artificial colors, natural flavors and/or inactive ingredients, many of which are found in tablets, capsules, liquid medications, candies and drinks. Starches, one of the most common drug and food additives are often genetically engineered and chemically treated, leaving many residues and/or composites on the final product. Many products also list natural flavors when they also contain chemicals and may be prepared and divided into sub types based on the amount and type of chemicals used in each preparation.

Chemical rings may be considered to provide surface area that may involve additional incubation and/or fermentation with different reaction times that may also be involved with different growth factors. These chemicals and/or rings may be involved with incubation and reactions while stored in foods, such as liquids, solids and semi-solids before they are ingested into the human body or any species for internal incubation and/or fermentation. The different incubation and/or fermentation mediums may be referred to as incubation and/or fermentation tanks. The incubation and/or fermentation phase or cycle and length of time the incubation occurs should also be considered, along with the growth and/or precursor complexes that may form chemical predispositions. For example, vegetable fed beef and/or pork shows less chemical incubation and/or contamination than hormone or chemically injected beef or pork. Bovine and porcine derived insulin are early examples that resulted in chemically manufactured proteins.

Starches are also genetically engineered and chemically treated. Once ingested, their parent compound and/or by-product incubation and/or fermentation may occur, completing and/or beginning another food additive-inactive ingredient complex continued cycle. This cycle may be correlated with the glucose cycle, glucose being a major precursor and/or toxin and/or toxin by-product substrate.

These food additive chemicals may result in substrates for drug therapies, since many of the prescribed drugs seem to fit into a reaction with the food additive chemicals, only targeting a portion of an incubated and/or fermented developed aggregated mass. The portion of the incubated developed aggregated mass may correlate with the molar mass value of a drug, food additive and/or inactive ingredient or inactive ingredient complex. Whether these food additives are artificial proteins, starches and/or both, they may result in an incubated and/or fermented complex that may show different growth characteristics, whether developing into what is viewed on a body part surface, such as a blood vessel, as a modified receptor site for drugs, or any other growth characteristic variations, such as a tumor on an organ or a blood bound protein complex that's serves as a carrier for blood toxin transport.

Some drug therapies use the terms viruses and bacteria to subcategorize treatments and different diseases, while others use the autonomic nervous system as the basis of many other therapies. Many of the drug rings and side groups correlate with the chemical structure of food additives. The term “inactive ingredient—food additive group complex” may be considered for the different types of diseases and conditions. Questions such as what type of inactive ingredient or food additive complex an individual acquires, where the inactive ingredient or food additive complexes are stored and how long have they incubated and/or fermented are important. When environmental gas and/or spore inhalation occurs, what inactive ingredient food additive complex (IIGFA) is activated, which inactive ingredient food additive complex result in extended and/or short reactions and which molar mass aggregates are activated are also important considerations. Using terms pertaining to chemical complexes, such as inactive ingredient food additive group complexes and molar mass aggregates, instead of organisms may also better fit the perception and/or point of views of many environmentalists and toxicologists when discussing these drug therapies.

Many individuals with natural food or vegetable allergies learn that it may not be the actual real natural vegetable or fruit that a person is allergic to, but rather by-products and break-up products that result from the fruit and vegetable breakdown and elimination of chemical toxins from any present inactive ingredients or food additive complexes. For example, some patients are diagnosed as being allergic to a vegetable such as corn or tomato, when in fact they are not. They may be highly allergic to genetically engineered, modified and/or other acid or artificially treated cornstarch and other derived or contaminated fruit and vegetable products, but not to the real natural product or vegetable. Some persons may also be allergic to the parent chemical compound, such as monosodium glutamate or processed chemical compound or complex or their by-product from degradation, but may not be allergic to the actual natural form of the actual fruit and vegetable they are eating.

The medical field continues to recognize the need for improving results from drug therapies, in addition to improving the efficacy of each drug therapy. The advantage is to decrease the amount and time at which the patient must be prescribed the drug to minimize any adverse effects that may occur. Increasing the amount of nutrients into a toxin layer where a drug penetrates may have significant therapeutic benefits and may be correlated with the healing of any damaged and/or artificially induced contaminated tissue growth. More importantly the patient is exposed to less polysaccharides and more nutrients. Since many foods contain similar additives to those found in drugs, a decrease in the amount of commonly used inactive ingredients may correlate with a decrease in the formation of additional toxin layer tissue, resulting in a thinner toxin layer, in turn allowing a quicker drug penetration, since the polysaccharide layers and complexes are what the drug penetrates.

Several drug formulations have been created in an attempt to increase the delivery and efficacy of drugs. The need for continued manufacturing of new drugs may result from the continued food consumption and drug re-administration of inactive ingredients to patients. By including less modified polysaccharide starches and non-genetically and chemically treated additives, the patient may be less likely to show adverse and side effects and in turn may experience quicker tissue healing. For example, non-GMO and chemically treated rice starch may help heal parietal cells in a patient's stomach.

The present invention generally relates to a drug composition. More specifically, the invention is a hypoallergenic nutrient delivery drug composition.

It is an object of the invention to provide a hypoallergenic nutrient delivery drug composition that provides healing of toxin tissue layers through increasing the amount of nutrients by administering naturally occurring non-genetically modified starches, oils, fruits and vegetables fillers with considerations to compatibility.

It is an object of the invention is to provide drug therapy using non-genetically engineered and non-chemically treated starches, oils, fruit and vegetable powder and/or extracts, as opposed to using chemically treated “polysaccharides” and/or complex modified artificial sugars, in addition to simple sugar precursors as opposed to real natural sugars. The treated starches (genetically and chemically) are oxidized, the effects of which are not known. Chemically modified and/or treated polysaccharides may form unwanted complexes with food additives such as monosodium glutamate and/or other related chemicals as seen in their chemical side groups.

Many patients being treated with current formulations may experience a rebound effect such as rebound headaches, when in fact many are allergic to the modified cornstarch, along with other inactive ingredients that may cause unwanted complexes and cause an allergic or allergenic response. The question remains whether this rebound effect may also relate to other organ responses and conditions.

It is an object of the invention to provide a hypoallergenic nutrient delivery drug composition that reduces adverse drug effects and increases the efficacy from other drugs currently taken by a user.

It is an object of the invention to provide a hypoallergenic nutrient delivery drug composition that penetrates into the toxin tissue layers along with targeting specific organs infected sites and replacing toxic substances from infected sites with reinserted unmodified nutritious substances.

What is really needed is a hypoallergenic nutrient delivery drug composition that provides healing of toxin tissue layers through increasing the amount of nutrients by administering naturally occurring non-genetically modified starches, oils, fruits and vegetables fillers that reduces adverse drug effects and increases the efficacy from other drugs currently taken by a user and that penetrates into the toxin tissue layers along with targeting specific organs infected sites and replacing toxic substances from infected sites with reinserted more nutritious substances.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

The hypoallergenic nutrient delivery drug composition is to provide inpatient and outpatient drug therapy using non-genetically engineered and non-acid or phosphate treated starches, as opposed to using modified organism treated polysaccharides and/or complex modified artificial sugars, in addition to simple sugar precursors as opposed to real natural sugars. Growing modified and modifying the non-genetically engineered and non-acid or phosphate treated starches after growth may also play an important role in the progression of diseases. Artificial genetic variations may correlate with artificial genetic predispositions. The genetically and chemically treated starches are oxidized, which may correlate in some way with antioxidants.

Phosphate treated starches and phospho-lipid formation may correlate with polysaccharide complexes and toxin layer development. Polysaccharides may show an unusual characteristic with regard to their storage and affinity for inactive ingredients and food additives, whether they are the parent compounds and/or degredative by-products. A polysaccharide as it sounds may correlate or show affinity to many sacs or immature toxin cells, which may correlate with saccharin and/or other related chemicals possibly involving toxin aggregation. Predispositions to cyanide (CN) bonds as seen in the monosaccharide glucosamine may play an important role in toxin cell development. The biological chemosynthesis reactions may serve as a basis for toxin cell aggregation and chemical ring attachment allowing more surface area for chemical reactions to occur, therefore creating an environment for active or non-active reactions or latencies. The concept of using less modified-polysaccharide fillers and/or other non-genetically modified inactive ingredients can apply to all drugs.

The hypoallergenic nutrient delivery drug composition may be utilized with a variety of drugs, such as glucophage (metformin), along with other oral antidiabetic or oral hypoglycemic drugs. In addition the nutrient delivery drug composition may be utilized with sinus, allergy, asthma, migraine headache and anti-coagulant (warfarin) drugs. Because of the chronic nature of diabetes and sinus headaches, a variety of derivative drugs have been added to the market that can only now adjust to any new toxin layers from continued consumption of inactive ingredients and/or food additives. For example, side group, chemical ring and bond re-arrangement is seen with each new drug. A continued layering of toxins may create the need for a new drug.

With the hypoallergenic nutrient delivery drug composition the continued use of genetically and chemically treated starches and proteins is minimized, avoided and replaced with natural and organic oils and starches, such as organically processed rice, real corn starch and many other real vegetable starches, excluding the use of phosphates, dextrins, absolute solvent extractions and synthetic acids, while using lime and other natural acids and antioxidants. In addition, pure organic natural honey, vegetable oils (carrier and essential oils) and/or other vegetable powders that contain the original nutrients and starches un-modified may also be used for gel caps, tablets and oral liquids. For example, pepper oils cause excretion and may break up polysaccharide synthetic starches, resulting in excretion of toxins via sweat and other areas. Corn kernels and endosperm have pure starch until treated and modified with synthetic acids and polysaccharides including dextrin and other similar chemicals which result in residues.

Although some patients may show more favorable results with methyl cellulose or carboxymethyl cellulose than with pre-gelatinized starches, glucose is stored for the next complex that may be formed with the pre-gelatinized starches. Although simple sugars may be more effective at times than polysaccharide complex sugars as seen in intravenous solutions of dextrose, real natural rice, corn, potato and other vegetable sugars and starches have nutrients that chemical rings in the active drugs can facilitate penetration into the toxin layers and/or cells for cell healing, toxin binding and correction. In addition, polysaccharides may create the need for osmosis through storage of continued food additive toxins and inactive ingredient groups and by-products, while unmodified vegetable starch nutrients may correct or calm reactions, replenishing and repairing the toxin cells. Synthetically induced poly sac (polysaccharide) fermentation may contribute to external and internal chemical toxin incubation and/or fermentation, as compared to organically natural non-acid chemically and non-genetically treated maize (corn), rice and other vegetable starches.

Antipsychotic, antidepressant and antiepileptic drugs may also be utilized with the hypoallergenic nutrient delivery drug composition along with many other drugs. It should also be noted that drug pharmacokinetics compared to food additive and inactive ingredient toxin-kinetics, including their parent compounds and by-product re-absorption, half-life and storage, may correlate with one another. It should also be noted that Heme ring complex A and B (hemoglobin) fits as a substrate with different inactive ingredient (IIG) and/or food additive precursors and parent compounds. Thus, hemoglobin transport of these toxins may occur, furthering transport of these toxins or partial masses to toxin aggregate locations, possibly effecting incubation and/or fermentation with continued toxin complex aggregation.

Patients that are asymptomatic to a particular formulation compared to patients that are allergic or symptomatic should not be ignored, as IIG-Food additive impacted fermentation may still occur in an asymptomatic individual and may contribute to later complications such as adverse effects and/or types of different growths or illnesses.

See Illustrations 1 and 2 regarding nutrient delivery drug composition and food modified and fruit and vegetable nutrient concentrated cells.

### Examples of Some Modified or Artificial Food Additives

These may serve as substrates for drugs, whether initially or in a complex after ingested with consideration to the chemical rings and side groups. Many may complex to form an aggregate mass and/or storage of other toxins. Most have been genetically modified (GMO) and later chemically washed with artificial synthetic acids and other chemicals such as dextrin like precursors, phosphates and/or chlorine such as hypochlorite as opposed to lime, ethanol and other real nutrients.

### Examples Include:

Glucose (ring form and
chain form) Sucrose
Dextrin or Dextrose
Modified Food Starch (D-Glucose units)
Maltdextrin from Corn
Corn Syrup
High Fructose Corn Syrup
Corn starch
Pre-gelatinized Corn Starch
Sodium Starch Glycolate
Monosodium Glutamate
Sodium Nitrite
Erythrobate (compare with Erythrocytes)
Dyes (Red-Yellow and
others) Xanthan Gum
Sodium Benzoate
Saccharin
Phenylalanine
All artificial proteins and amino acids
Aspartame
Guanylate salts
Modified chemically treated oils (absolute solvent extractions and hydrogenated)

### Examples of Some GMO and/or Chemically Treated Inactive Ingredients

Cross-utilization accumulation may occur with food additive and drug excipients such as Xanthan gum, color dyes, corn syrups and starches. Some common cross-utilized additives may show correlations with certain aggregates or masses. Examples include:
Corn Starch
Pre-gelatinized Corn Starch
Hydroxymethylcellulose
Methyl cellulose
Sodium Benzoate
Xanthan Gum
Lactose monohydrate
Polyethylene glycol
Microcrystalline cellulose
Povidone
FDC Colors aluminum lakes
Silicon dioxide
Sodium lauryl sulfate
Titanium dioxide

### Examples of Current Drug Formulations and Compounds

### Example Template:

Drug -----X------ Which can be a homeopathic ingredient
Binder ---x----- (solution binders: gelatin, cellulose and/or derivatives (polyvinylpyrrolidone, starch, sucrose and polyethylene glycol)
Dry binders: cellulose, methyl cellulose, polyvinyl (pyrrolidone and polyethylene glycol)
Disintegrant --x- (Sodium Starch glycolate, cross linked- Sodium carboxymethyl cellulose a.k.a. crosscarmellose))
Filler and Diluent -X- (Plant cellulose, vegetable fats and oils (in capsules, lactose, sucrose, glucose (mannitol, sorbitol, calcium carbonate (and magnesium stearate)))
Preservative ---X-- (Vitamin A, E, C, retinyl palmitate and selenium (which are also synthetic antioxidants) (Amino acids and sodium citrate) (methyl paraben and propyl paraben))

### Example Drug:

### Glucophage (Metformin HCL SOOMG Tablets)

Active Ingredient: Metformin HCL
Inactive Ingredient: microcrystalline cellulose, magnesium stearate, povidone, hydroxypropyl cellulose and polyethylene glycol. (may vary with different generics)
Example Drug: Warfarin
Active Ingredient: Pregelatinized corn starch along with others (see drug)

### Examples of Pure Organic Carrier and/or Essential Oils, Vegetable Powder &/or Extracts & Non-GMO-Non Chemically Treated & Washed (NCTW) Starches in Place of Current Standard Excipients

Pure organic oils and vegetable powder and/or extracts that are free of GMO, synthetic acids, salts, chlorine treatments, absolute solvent extractions and other chemical treatments and washes may show less drug and food inactive ingredient adverse and/or side effects, increase a drug's pharmacological effect, decrease the need for increasing the dose of the drug and need for switching the drug. In addition, vegetarian fed pork, beef, lamb and other similar extracts that are free from GMO, synthetic acids and similar salt treatments/chemicals, may also be included when appropriate and desired, such as vegetarian fed and treated selections.

### Examples of New Hypoallergenic Nutrient Delivery Drug Formulations

### Example Template 1: Capsule or Tablet

### Ingredients:

Drug -----X------
Binder ---X----- (Real non-GMO and non-chemically washed or treated (Vegetable starches—rice, corn, potato and
others) (Bean X powders, pepper X
powders and others)—individual starches
or a mixture of starches
(Habanera, jalapeno and red chili powders))
Filler -X------- (Bean, garlic, onion, celery and other vegetable powders, in addition to fruit powders
Preservative -X--- (lime and/or lemon juice and/or oils
and)
(Vitamin E oil along with others)

### Example Template 2: Capsule

Ingredients: Drug ---X-----
Filler ---X---- (Bean, garlic, onion, celery and other vegetable powders in addition to fruit powder)
(Avocado oil, Almond oil, apricot oil, pure corn oil) (Grape seed oil, peanut oil, safflower oil, sesame oil)
(Pure soy oil, sunflower oil, walnut oil, Habanera oil)
(Cheyenne, jalapeno and other pepper oils), carrot oil
Preservative: lime or lemon juice/oils and Vitamin E oil along with Others (honey)

### Sustained Nutrient Drug Delivery Formulations

The nutrient delivery drug formulation involves organic starches, vegetable and fruit powder and/or extracts and oils that have not been genetically modified (non-GMO), chemically treated or washed (non-NCTW) and extracted with absolute solvents, such as dextrins, phosphates, chlorine (hypochlorite), hexane, acetone and other synthetic acids, bases, salts and solvents. The use of steam extracted organic oils such as key lime, lime, orange, Vitamin E and other oils which also have antioxidant properties in conjunction with other alkaline and acid vegetable and fruit powders may also be used in place of chemically treated and genetically modified common inactive ingredients and/or excipients. Individual oils with the above requirements may also be used, while unique ratios of mixtures of oils and/or fruit and vegetable powder and/or extracts and starches may also be used.

### Example of Oils, Fruit & Vegetable Powders and Starches for Formulation Mixtures

Corn, Avocado, Sesame, Peanut, Safflower, Apricot, Wheat Germ, Sunflower, Almond, Sunflower seed, Hazelnut, Olive oil, Asparagus, Oat, Chestnut, Coconut, Walnut, Carrot, Orange, Lime, Lemon, Flaxseed extract, Vitamin E, Jalapeno, Habanera, Black pepper and other Red Chili oils (oils that use cold pressed, expeller methods and/or Ghani method of Extraction).

Starches: Non-GMO-NCTW: Corn Starch, Rice Starch, Arrowroot, Arracacha, Barley, Oat, Millet, Rye, Banana, Potato, Bean and Pea starches.
Optional When Appropriate to Use an Extract: Vegetable and Fruit Powder Extracts: Apple powder extract, Grape-seed extract, Pomegranate extract, Mangosteen extract, Orange extract, Blueberry extract, Mulberry Fruit extract, Sweet Honeysuckle extract, Black Current extract, Orange peel extract, Lime Peel extract, Tomato extract, Vegetable juice extract, Parsley extract, Cilantro extract, Raw Spinach extract, Celery extract, Garlic extract, Barley Grass extract, Green Bean extract, Beet extract, Lettuce extract, Carob extract, Carrot root extract, Dried pea juice, soy bean extract, deflated wheat germ extract, pure soy extract (mixed tocopherols) and Broccoli extract.
Optional Vegetarian Fed Beef, Pork, Porcine, Lamb and Fish Extracts: Bovine liver, Bovine Kidney, Bovine Prostate, Bovine fat extract, Bovine adrenal extract, Bovine Thymus extract and Bovine bone extract.

### Broad Spectrum Nutrient Delivery Formulation

(MIXTURE VARIATIONS): FV=Fruit and Vegetable

### Consituents:

1. MONO-PEPPER OIL MIXTURE
2. 01-PEPPER OIL MIXTURE
3. TRI-PEPPER OIL MIXTURE
4. POLY-PEPPER OIL MIXTURE
5. POLY-PEPPER OIL—ERB MIXTURE
6. POLY-FV-OILMIXTURE
7. MONO-NON-GMO-NCTW-STARCH (Rice—Corn—Potato—Vegetable)
8. 01-NON-GMO-NCTW-STARCH MIXTURE
9. TRI-NON-GMO-NCTW-STARCH MIXTURE
10. MONO-FV-POWOER EXTRACT
11. 01-FV-POWOER EXTRACT
12. TRI-FV-POWOER EXTRACT
13. POLY-FV-POWOER EXTRACT
   Poly-FV-Oil Mixture 1: mix 40 ml of each to give a total of 480 ml
Sesame
Avocado
Black Pepper
Vitamin E Apricot Almond
Carrot
Wheat Germ
Lime oil Key lime Orange
Garlic
   Poly-FV-Oil-Mixture 2: mix 37.5 ml of each. Use 10 ml each of pepper oil for 480 ml
Sesame
Avocado-Black Pepper
Vitamin E Apricot Almond
Carrot
Wheat Germ
Lime oil
Key lime
Orange
Garlic
Habanera: 10 ml Jalapeno: 10 ml Cheyenne 10 ml
   Poly FV Powders and/or Extract (Non-GMO-NCTW): mix 2.3 Grams of equal parts for a total of 48.3 grams:
Grape seed
Pomegranate
Mangosteen
Orange Blue Berry Mulberry Fruit
Sweet Honey Suckle
Black Current Orange Peel Lime Peel
Key lime Peel
Tomato
Broccoli
Carrot
Parsley
Cilantro Papaya Beet
Oat Bran
Rice
Spinach

### (Poly FV Oil or Poly-Pepper Oil General Formula (#_Capsule) Quantity: 100 Capsules

### Ingredients:

### Active Ingredient

| | | |
|---|---|---|
| Poly FV Powder | .5 | grams |
| Poly FV Oil | q.s. 50 | ml |

Calculate (may use Silicon Dioxide if needed)
1. Triturate active ingredient with Poly FV powder thoroughly.
2. Slowly add small amounts of oil and mix to form a paste
3. Add remaining oil in small increments
4. Transfer 0.5 ml to each capsule (#1) with a syringe

### Formulation Examples Used: Glipizide SMG Capsules (#1 Capsule)

Quantity: 100

### Ingredients:

| | |
|---|---|
| Glipizide USP | .5 grams |
| Cilantro Powder | 25.34 grams |

Mixing instructions: Triturate powders and encapsulate in #1 capsules.

Sufficient lime, lemon and orange may be used as additional preservatives with or without Vitamin E. However, Poly-FV Extract may provide sufficient anti-oxidant properties for at least 180 days or more, depending on concentrations and percentages that are needed for longer shelf lives.

### Glipizide 10 Mg Capsules (#1 Capsule)

Quantity: 100

### Ingredients:

| | |
|---|---|
| Glipizide USP | 1 gram |
| Cilantro Powder | 25.34 grams |

Mixing instructions: Triturate powders and encapsulate in #1 capsules.

### Glipizide SMG Capsules (#1 Capsule)

Quantity: 100

### Ingredients:

| | | |
|---|---|---|
| Glipizide USP | .5 | grams |
| Rice Starch - Non GMO-NCTW | 25.34 | grams |
| Lime oil | QS 34 | ml |
| Poly FV Oil #1 | QS 34 | ml |

Mixing instructions: Triturate powders and oils, and encapsulate in #1 capsules.

### Metformin HCL SOOMG Capsules (#1 Capsule)

Quantity: 100

### Ingredients:

| | |
|---|---|
| Metformin USP | 50 grams |
| Poly-FV-Oil #1 | 23.5 ml |
| Poly-FV-Oil #2 | 23.5 ml |

Use 00 Capsule that holds 0.95 mi.
Bring total volume to 100 ml with oil. Slowly add a small amount of Poly- FV Oil #1 and mix to form a paste. Add remaining Polly-Pepper Oil in small increments and mix.

### Loratidine 10 Mg Capsules (#1 Capsule)

Quantity: 100

### Ingredients:

| | |
|---|---|
| Loratidine USP | 1 gram |
| Poly-FV-Powder | 20 grams |
| Poly-Pepper Oil | 68 mls. |

### Loratidine 10 Mg Capsules (#1 Capsule)

Quantity: 100

### Ingredients:

| | |
|---|---|
| Loratidine USP | 1 gram |
| Poly-FV-Oil | 20 grams |
| Poly-Pepper Oil | 68 ml |

Mixing directions: Triturate Loratadine, USP and Poly-FV-Extract. Slowly add a small amount of PolyPepper Oil and mix to form a paste. Add remaining Polly-Pepper Oil in small increments and mix. Transfer 0.68 ml into each #0 or #1 or the appropriate capsule using a syringe. Fill to top.

### Ibuprofen Capsules 400 Mg (#0 or 1 Capsule)

Quantity: 100

### Ingredients:

| | |
|---|---|
| Ibuprofen USP | 40 gram |
| Poly-FV-Powder | 5 grams |
| Poly-FV-Oil #1 | 18.8 ml |
| Poly-Pepper Oil | 18.8 ml |

Calcium carbonate Heavy, USP may be used if needed. Although alkaline vegetable mixtures maybe sufficient, they should be adjusted accordingly.

Hydrocodone Bitartrate 10 Mg and Acetaminophen 325 Mg (#0 Capsule)
Quantity: 100

### Ingredients:

| | | |
|---|---|---|
| Hydrocodone Bitartrate USP | 1 | gram |
| Acetaminophen USP | 32.5 | grams |
| Poly-FV-Powder | 5 | grams |
| Poly-FV Oil #2 | 13 | ml |

Example Drug: Current formulation

### Glucophage (Metformin HCL SOOMG Tablets)

Inactive Ingredient: microcrystalline cellulose, magnesium stearate, povidone, hydroxypropyl cellulose, polyethylene glycol

### Organiformin (New Formulation)

### Metformin HCL 500 Mg Capsules (#00 Capsule)

Quantity: 100

### Ingredients:

| | |
|---|---|
| Metformin USP | 50 grams |
| Cilantro Oil-Powder mixture | 23.5 ml |
| Organic Oil Mixture Rx | 23.5 ml |

Note: Organic oil mixture contains equal parts of expeller or cold pressed Avocado, Carrot and Almond oils.
Use 00 Capsule that holds 0.95 mi.
Bring total volume to 100 ml with Oil. Slowly add a small amount oil and mix to form a paste. Add remaining Oil in small increments and mix.

### Organiprofen Plus

### Ingredients:

| | | |
|---|---|---|
| Loratidine USP | 1 | gram |
| Ibuprofen, USP | 40 | grams |
| Calcium Carbonate Heavy, USP | X | grams |
| Carrot Powder | 10 | grams |
| Cilantro Powder | 10 | grams |
| Organic Oil Mixture Rx-PO | 68 | ml |

Note: Organic oil mixture Rx-PO contains 5% pepper and Onion oils.
Mixing directions: Triturate Loratadine, USP and Ibuprofen. Slowly add a small amount of Organic oil mixture and mix to form a paste. Add remaining Organic Oil Mixture Rx in small increments and mix. Transfer 0.68 ml into each #0 or #1 or the appropriate capsule using a syringe. Fill to top.

Note: Non GMO-Non NCTW Rice or Cornstarch may be exchanged and substituted for lactose or any of the synthetic modified or chemically washed fillers and/or binders. In addition, other powder and/or oils may be used in conjunction or individually or along with a nutrient delivery composition to adjust the ratio of current excipients and drugs in formulations. Additional drugs may be substituted with the same standard formulas with some variations and minor adjustments.

The term IIG Gap (inactive ingredient gap) is used to determine the percentage gap that represents the amount of fillers used and the % reference for including more nutritious fillers:

### Example

Total tablet or capsule weight (TCW)−Weight of active ingredient (Aw)=IIG Gap

### Drug X:

Total Tablet weight=100 mg
Total active drug=30 mg
IIG Gap=70%

The following are samples of over 60 patients who received re-formulated prescriptions that were compounded and dispensed at the Medicine Shoppe Pharmacy 1435 State Street Santa Barbara, Calif. 93101 within the following date ranges: 2010 through 2012

### Trial #1

Drug (active pharmaceutical ingredient): Loratidine capsules in:
Vehicle: Expeller pressed or cold pressed non-hydrogenated almond oil, sesame oil, avocado oil, coconut oil and/or olive oil with or without infused chili olive oil 0.25-0.5 ml/capsule with optional: Non genetically modified, non-genetically engineered or non-chemically treated rice starch, potato starch, or pea starch and cellulose from blueberry, carrot, peas, celery, beans and brussels. Vehicle also includes a vitamin E oil preservative.
Strength: 10 mg
Results: Substantial improvement of inflammation symptoms such as sinus drainage, headache, rash and pain.
Dates:
Apr. 28, 2011 #20 capsules
Aug. 26, 2011 #10 capsules
Aug. 29, 2011 #10 capsules
Sep. 15, 2011 #30 capsules
Sep. 26, 11 #30 capsules
Sep. 27, 2011 #10 capsules
Sep. 29, 2011 #15 capsules
Oct. 11, 2011 #10 capsules
Oct. 14, 2011 #10 capsules
Dec. 14, 2011 #60 capsules

### Trial #2

Drug (active pharmaceutical ingredient): Ibuprofen capsules in:
Vehicle: Expeller pressed or cold pressed non-hydrogenated almond oil, sesame oil, avocado oil, coconut oil and/or olive oil with or without infused chili olive oil 0.5 ml/capsule with optional non-genetically modified, non-genetically engineered and non-chemically treated rice starch, potato starch or peas starch and cellulose from blueberry, carrot, peas, celery, beans and brussels. Vehicle also includes a vitamin E oil preservative.
Strength: 200 mg or 400 mg
Results: Substantial improvement of inflammation symptoms such as sinus drainage, headache and rash.
Dates:
Jan. 13, 2010 #10 capsules
Apr. 26, 2011 #10 capsules
Aug. 17, 2011 #50 capsules
Sep. 26, 2011 #30 capsules

### Trial #3

Drug (active pharmaceutical ingredient): Acetaminophen capsules in:
Vehicle: Expeller pressed or cold pressed non-hydrogenated almond oil, sesame oil, avocado oil, coconut oil and/or olive oil with or without infused chili olive oil 0.5 ml/capsule with optional non-genetically modified, non-genetically engineered or non-chemically treated rice starch, potato starch or pea starch and cellulose from blueberry, carrot, peas, celery, beans and brussels. Vehicle also includes a vitamin E oil preservative.
Strength: 500 mg
Results: Substantial improvement of inflammation symptoms such as headache and pain.
Dates:
Jan. 6, 2011 #10 capsules
Oct. 25, 2011 #10 capsules
Jul. 12, 2011 #10 capsules
Sep. 21, 2011 #10 capsules
Sep. 30, 2011 #30 capsules
Oct. 25, 2011 #30 capsules

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A chemical composition, consisting of:
loratidine;
a selected one or more of an expeller pressed and a cold pressed oil;
a selected one or more of a non-genetically modified, non-genetically engineered and a non-chemically treated starch; and
a selected one or more of a non-genetically modified, non-genetically engineered and a non-chemically treated cellulose.

2. The chemical composition according to claim 1, wherein said chemical composition includes approximately 10 mg of loratidine.

3. The chemical composition according to claim 1, wherein said selected one or more of an expeller pressed and a cold pressed oil is almond oil.

4. The chemical composition according to claim 1, wherein said selected one or more of an expeller pressed and a cold pressed oil is sesame oil.

5. The chemical composition according to claim 1, wherein said selected one or more of an expeller pressed and a cold pressed oil is avocado oil.

6. The chemical composition according to claim 1, wherein said selected one or more of an expeller pressed and a cold pressed oil is coconut oil.

7. The chemical composition according to claim 1, wherein said selected one or more of an expeller pressed and a cold pressed oil is olive oil.

8. The chemical composition according to claim 7, wherein said selected one or more of an expeller pressed and a cold pressed oil is infused chili olive oil.

9. The chemical composition according to claim 8, wherein said infused chili olive oil is in the approximate range of 0.25 to 0.50 ml.

10. The chemical composition according to claim 1, wherein said selected one or more of an expeller pressed and a cold pressed oil is vitamin E oil preservative.

11. The chemical composition according to claim 1, wherein said selected one or more of a non-genetically modified, a non-genetically engineered and a non-chemically treated starch is rice starch.

12. The chemical composition according to claim 1, wherein said selected one or more of a non-genetically modified, a non-genetically engineered and a non-chemically treated starch is potato starch.

13. The chemical composition according to claim 1, wherein said selected one or more of a non-genetically modified, a non-genetically engineered and a non-chemically treated starch is pea starch.

14. The chemical composition according to claim 1, wherein said selected one or more of a non-genetically modified, a non-genetically engineered and a non-chemically treated cellulose is blueberry cellulose.

15. The chemical composition according to claim 1, wherein said selected one or more of a non-genetically modified, a non-genetically engineered and anon-chemically treated cellulose is carrot cellulose.

16. The chemical composition according to claim 1, wherein said selected one or more of a non-genetically modified, a non-genetically engineered and anon-chemically treated cellulose is pea cellulose.

17. The chemical composition according to claim 1, wherein said selected one or more of a non-genetically modified, a non-genetically engineered and a non-chemically treated cellulose is celery cellulose.

18. The chemical composition according to claim 1, wherein said selected one or more of a non-genetically modified, a non-genetically engineered and a non-chemically treated cellulose is bean cellulose.

19. The chemical composition according to claim 1, wherein said selected one or more of a non-genetically modified, a non-genetically engineered and a non-chemically treated cellulose is Brussels cellulose.

20. The chemical composition according to claim 1, wherein said chemical composition is encased in a capsule.
